(19) United States
(12) Patent Application Publication
(10) Pub. No.: US 2023/0116113 A1
(43) Pub. Date: Apr. 13, 2023

(54) USE OF COMPOUNDS THAT ARE ABLE TO CROSS-LINK THE EXTRACELLULAR MATRIX FOR PREVENTING OR INHIBITING THE MIGRATION OF CANCER CELLS

(71) Applicant: CAMBRIDGE ENTERPRISE LIMITED, Cambridgeshire (GB)

(72) Inventors: Melinda Jane DUER, Cambridge (GB); Uliana BASHTANOVA, Cambridge (GB); Rakesh RAJAN, Cambridge (GB)

(21) Appl. No.: 17/799,320

(22) PCT Filed: Feb. 12, 2021
(86) PCT No.: PCT/GB2021/050345

§ 371 Date: Aug. 12, 2022

(30) Foreign Application Priority Data
Feb. 13, 2020 (GB) 2001963.4

(51) Int. Cl. A61K 31/352; A61K 31/11; A61K 31/525; A61K 31/5415; A61K 31/132; A61K 31/728; A61K 41/00; A61P 35/00

(52) U.S. Cl. CPC A61K 31/352; A61K 31/11; A61K 31/525; A61K 31/5415; A61K 31/132; A61K 31/728; A61K 41/00; A61P 35/00

(57) ABSTRACT

The present invention relates to the field of cancer progression. The invention provides in vivo methods for preventing or inhibiting the migration of cancer cells away from the site of a tumour or a resected tumour, thus inhibiting invasion and metastasis, by contacting all or part of the tumour or resected tumour, or all or part of the vicinity of the tumour, with an agent which impedes the migration and/or proliferation of cancer cells, such as a cross-linking agent.

The present invention relates to the field of cancer progression. The invention provides in vivo methods for preventing or inhibiting the migration of cancer cells away from the site of a tumour or a resected tumour, thus inhibiting invasion and metastasis, by contacting all or part of the tumour or resected tumour, or all or part of the vicinity of the tumour, with an agent which impedes the migration and/or proliferation of cancer cells.

Despite developments in cancer therapy, cancer metastasis remains a significant challenge in cancer treatment. Cancer cell transition to a migrating or metastatic phenotype and the survival of migrating cancer cells are well documented to depend on the biochemical composition and biomechanical properties of the host tissue and tumour extracellular matrix. Migrating cancer cells may invade other regions of the host tissue in which the primary tumour is sited or move to other tissues, creating metastatic tumours which are difficult to treat.

For cancer cells to metastasize, they must migrate through the extracellular matrix surrounding the tumour. Although the extracellular matrix composition is highly dynamic and specialized to each tissue, in general it consists of collagens, hyaluronic acid, and tissue-specific glycoproteins and proteoglycans [T. R. Cox, J. T. Erler, Disease Models and Mechanisms 4 (2011) 165]. The major components in all tissues are either collagens or hyaluronic acid (sometimes called hyaluronan or hyaluronate) or both. Collagens and hyaluronic acid are the main structural elements of the extracellular matrix and have important roles in cell biology. Hyaluronic acid regulates cell adhesion, motility, growth and differentiation through interactions with cell surface receptors such as CD44, and through being the primary organizer of extracellular matrix proteoglycans that also interact with cells. Fibrillar collagens (collagen types I, II, III and V), where they exist, are an integral component of (cancer) cell migration, providing tracks for cells to move through tissues [C. Walker, et al., Int. J. Molec. Sci. 19 (2018) 32]. Non-fibrillar collagen type IV is the main structural component of the specialized extracellular matrix, basement membrane, and has a number of cell signalling roles.

Over- or under-expression of structural extracellular matrix components or modifiers in the tumour setting has significant correlation with tumour prognosis. For instance, many cancers are associated with deposition of collagen type I or IV in the surrounding ECM or enzymes that modify collagens [S. Xu, et al., J. Trans. Med. 17 (2019) 302]. These cancers include breast cancer [T. Oskarsson, The Breast, 22 (2013) S66), pancreatic ductal adenocarcinoma [M. Weniger, K. C. Honselmann, A. S. Liss, Cancers, 10 (2018) 32], lung cancer [G. Burgstaller, et al., Eur. Resp. J. 50 (2017) 1601805] and liver cancer [R. Zhang, et al., BMC Cancer, 18, (2018) 901] and where increased collagen deposition occurs, tumours often have poorer prognosis [S. Xu, et al., J. Trans. Med. 17 (2019) 302]. Hyaluronic acid is hypothesised to promote survival of metastatic cancer cells and tumours that over-express hyaluronic acid are also often associated with poorer prognosis [P. Lu, et al., J. Biol. Chem. 196 (2012) 395].

Cancer cell migration requires degradation and/or deformation of the structural components of the tumour and host tissue extracellular matrix to generate space for the migrating cancer cell bodies to pass through [K. Wolf, P. Friedl, Trends in Cell Biology 21 (2011) 736]. Thus, expression of metalloproteases (MMPs) that degrade collagens and hyaluronidases that degrade hyaluronic acid are a frequent feature of metastatic tumours [C. Bonnans, et al., Nature Reviews Molec. Cell Biol. 15 (2014) 786; C. O. McAtee, et al., Adv. Cancer Res. 123 (2014) 1].

The clear roles of collagens and hyaluronic acid in cell adhesion, migration and tumour progression mean that it is useful to stratify cancers according to whether the host or tumour tissue is hyaluronic acid- or collagen-rich or contains significant percentages of both. For instance, brain glioblastoma multiforme cancer occurs exclusively in the brain and occasionally central nervous tissues, where the extracellular matrix is rich in hyaluronic acid (and low in collagen type I). Osteosarcomas occur primarily in bone, where the organic part of the extracellular matrix is rich in collagen type I (and low in hyaluronic acid).

As described above, to migrate, cancer cells must move through the extracellular matrix (ECM) that surrounds the tumour. The inventors have now demonstrated that cancer cell migration can be prevented or hindered by physically impeding the movement of cancer cells away from the site of the cancer. This is done by chemically altering—in vivo—the extracellular matrix around a tumour to generate an in vivo environment where cancer cells are impeded from moving, for example by increasing the molecular density or biopolymer entanglement within the in vivo extracellular matrix around a tumour or chemically cross-linking in the extracellular matrix in and around a tumour. The inventors have established a 3D model of the brain extracellular matrix and demonstrated, using six different cross-linking strategies, that cancer cell proliferation and migration can be significantly inhibited in this way. They have further established a 3D model of the osteosarcoma tumour environment and demonstrated, using two different crosslinking strategies that cancer cell proliferation and migration can also be significantly inhibited in this way.

It is one object of the invention, therefore, to provide an in vivo method of preventing or inhibiting the migration of cancer cells away from the site of a tumour or site of a resected tumour. In particular, it is one object of the invention to prevent cancer cells which might remain after the resection of a tumour from invading further into the subject. In particular, it is one object of the invention to inhibit proliferation of cancer cells which might remain after the resection of a tumour. In particular, following surgical resection of brain glioblastoma multiforme, tumour regrowth is known to occur in nearly all patients at the original tumour site with 3-6 months; cells derived from this tumour also often metastasize to other parts of the brain. It is an object of the invention to provide methods to prevent such tumour regrowth or metastasis in brain glioblastoma multiforme, and in other tumours.

In one embodiment, the invention provides an in vivo method of preventing or inhibiting the migration of cancer cells away from the site of a tumour or site of a resected tumour on or in a subject, the method comprising contacting all or part of:
(i) the tumour,
(ii) the site of the resected tumour, and/or
(iii) the vicinity of the tumour or site of resected tumour, with a composition comprising an agent which impedes the movement and/or proliferation of cancer cells, thus preventing or inhibiting migration of cancer cells away from the site of the tumour or site of the resected tumour.

The invention also provides a composition comprising an agent which impedes the movement and/or proliferation of cancer cells, for use in a method of preventing or inhibiting the migration of cancer cells away from the site of a tumour or site of a resected tumour on or in a subject, thus preventing or inhibiting migration of cancer cells away from the site of the tumour or site of the resected tumour, the method comprising contacting all or part of:
(i) the tumour,
(ii) the site of the resected tumour, and/or
(iii) the vicinity of the tumour or site of resected tumour, with the composition.

The invention also provides the use of an agent which impedes the movement and/or proliferation of cancer cells, in the manufacture of a composition for preventing or inhibiting the migration of cancer cells away from the site of a tumour or site of a resected tumour on or in a subject, thus preventing or inhibiting migration of cancer cells away from the site of the tumour or site of the resected tumour, the method comprising contacting all or part of:
(i) the tumour,
(ii) the site of the resected tumour, and/or
(iii) the vicinity of the tumour or site of resected tumour, with the composition.

In a further embodiment, the invention provides an in vivo method of inducing dormancy or differentiation in cancer cells in a tumour or site of a resected tumour on or in a subject, the method comprising contacting all or part of:
(i) the tumour,
(ii) the site of the resected tumour, and/or
(iii) the vicinity of the tumour or site of resected tumour, with a composition comprising an agent which impedes the movement and/or proliferation of cancer cells, thereby inducing cancer cell dormancy or tumour dormancy, or cancer cell differentiation, in the cancer cells or in the tumour.

Preferably, the agent is one which is capable of:
(a) (i) cross-linking cancer cells in the tumour, and/or
   (ii) cross-linking extracellular matrix in and/or around the tumour, and/or
   (iii) cross-linking cancer cells to the extracellular matrix in and/or around the tumour; and/or
(b) increasing the viscosity and/or molecular entanglement of the extracellular matrix in and/or around the tumour or site of the resected tumour, preferably by forming a secondary network in the extracellular matrix in and/or around the tumour.

The method of the invention is carried out in vivo, i.e. on or in the human or animal body. Preferably, the subject is a mammal, more preferably a human, mouse, rat, horse, pig, cow, sheep, goat. Most preferably, the subject is human. In some embodiments, the subject is a non-human mammal. The human may, for example, be 0-10, 10-20, 20-30, 30-40, 40-50, 50-60, 60-70, 70-80, 80-90, 90-100 or above 100 years old.

The tumour may be a benign, pre-malignant or malignant tumour. The tumour may be a primary or secondary tumour. The tumour is preferably a solid tumour. The tumour comprises cancer cells.

In some embodiments, the tumour is one whose size or carcinogenic (e.g. invasive) capabilities has previously been reduced. For example, the tumour may be one which has previously been at least partially resected (removed). Alternatively, or additionally, the tumour may be one which has previously been treated with another anti-tumour treatment, e.g. chemotherapy, immunotherapy or radiation, or a combination thereof.

The tumour may be one which is characterised by the extracellular matrix (ECM) of its immediate environment, vicinity or adjacent tissues.

In some embodiments, the tumour is located within a hyaluronic acid-rich environment or tissue, or in a location wherein hyaluronic acid content is elevated or wherein hyaluronic acid synthesis is increased. A hyaluronic acid-rich environment is one wherein the hyaluronic acid content in the ECM is sufficient to form a non-collagenous scaffold when fibrillar collagen is present or when fibrillar collagen is not present; or wherein hyaluronic acid content is elevated due to disease or injury.

In some embodiments, the environment or tissue is one wherein the concentration of hyaluronic acid is 0-1%, 1-2%, 2-3%, 3-4%, 4-5%, 5-6%, or above 6% of the wet weight of total ECM. In some embodiments, the environment or tissue is one wherein the concentration of hyaluronic acid is 0.5-1.0%, 1.0-1.5%, 1.5%-2.0%, 2.0%-2.5%, 2.5%-3.0%, 3.0%-3.5%, 3.5%-4.0%, 4.0%-4.5%, 4.5%-5.0%, 5.0%-5.5%, 5.5%-6.0% or above 6.0% of the wet weight of total ECM.

Hyaluronic acid-rich environments or tissues include the brain, cartilage, synovial fluid, skin, vitreous body of the eye and various tumours.

The proportions of hyaluronic acid reported in normal brain ECM are 10% hyaluronic acid by dry weight; this corresponds to 3% wet weight, assuming 70% brain tissue is water.

In some embodiments, the environment or tissue is a brain tissue wherein the concentration of hyaluronic acid in the brain tissue ECM is 3-4 wt %, preferably about 3.5 wt % hyaluronic acid of the wet weight of total ECM. In other embodiments, the hyaluronic acid-rich environment or tissue is a brain tissue wherein the concentration of hyaluronic acid in the brain tissue ECM is 4-6 wt %, preferably 4.5-5.5 wt %, more preferably about 5 wt % hyaluronic acid by wet weight.

Glioblastomas are known to over-express hyaluronases (i.e. enzymes which cut hyaluronic acid into smaller molecular units). This decreases the viscosity and molecular entanglement of hyaluronic acid around the tumour; small hyaluronic acid units may be washed away. Thus in embodiments wherein the tumour is a glioblastoma multiforme, the hyaluronic acid proportion may be equal to or below 1% in the brain ECM wet weight.

In other embodiments therefore, the environment or tissue is a brain tissue wherein the concentration of hyaluronic acid in the brain tissue ECM is 1.5-2.5 wt %, preferably about 2 wt % hyaluronic acid. of the wet weight of total ECM.

Increase in content or synthesis of hyaluronic acid may be tested for by measuring uronic acid content after hydrolysis in concentrated sulphuric acid, RNA analysis or ELISA or ELISA-like assays of tumour material (from biopsy or surgery) to quantify hyaluronic acid synthase (HAS1, HAS2, HAS3) expression or hyaluronic acid assay of tumour material; immuno-confocal imaging (staining for HAS1, HAS2, HAS3), or histochemical staining for hyaluronic acid on tumour sections [Cowman et al., 2015. Front Immunol. 6: 261]. The most sensitive, specific, and accurate methods for determination of HA content are based on enzyme-linked sorbent assays. In some patients, hyaluronic acid over-expression may be detected by analysing the soluble hyaluronic acid in blood through e.g. ELISA-like assays on blood samples.

Environments or tissues where hyaluronic acid synthesis is increased include in pancreatic ductal adenocarcinomas (PDACs), lung carcinoma, ovarian cancer and prostate cancer.

In other embodiments, the tumour is located in an environment or tissue where fibrillar collagen (collagen types I, II, III and V) form a scaffold or collagen IV forms a net, or these collagens are over-expressed/over-produced due to disease or injury.

An environment or tissue where fibrillar collagens (collagen types I, II, III and V) or collagen IV is overexpressed is one wherein levels of RNA or protein expression for one or more of these collagen types is found to be upregulated/overproduced compared to the normal host environment or tissue. Such levels may be measured by PCR, immunohistochemistry, proteomics analysis or specific protein analysis, e.g. by ELISA assays. Alternatively, collagen over-expression may be detected by an increase in soluble collagen or collagen fragments or hydroxyproline (a key component of collagens rarely found in other proteins) present in blood.

Environments or tissues wherein fibrillar collagens or collagen IV form the scaffold/net including most of body tissues, excluding brain tissues and body fluids, but including synovial fluid. Fibrillar collagens and collagen IV may be over-expressed in solid tumours by cells involved in the cancer niche, including cancer-associated fibroblasts, immune cells and cancer cells and has been found to be a feature in bone, lung, ovarian, breast and colorectal cancers, chondrosarcomas, PDACs, head and neck squamous cell carcinoma (HNSCC), osteosarcoma, skin cancer, thoracic cancer, respiratory cancer, tissue fibrosis and glioblastoma.

Preferably, the tumour is located within an environment or tissue wherein the concentration of collagen is 40 dry wt % to 95 dry wt %, more preferably 60 dry wt % to 90 dry wt %.

Cancers are classified by the type of cell that the tumour cells resemble and is therefore presumed to be the origin of the tumour. These types include:
Carcinoma: Cancers derived from epithelial cells. This group includes many of the most common cancers and include nearly all those in the breast, prostate, lung, pancreas and colon.
Sarcoma: Cancers arising from connective tissue (i.e. bone, cartilage, fat, nerve), each of which develops from cells originating in mesenchymal cells outside the bone marrow.
Germ cell tumour: Cancers derived from pluripotent cells, most often presenting in the testicle or the ovary (seminoma and dysgerminoma, respectively).
Blastoma: Cancers derived from immature “precursor” cells or embryonic tissue.

Brain and nervous system cancers include Astrocytoma, Brainstem glioma, Pilocytic astrocytoma, Ependymoma, Primitive neuroectodermal tumour, Cerebellar astrocytoma Cerebral astrocytoma, Glioma, Medulloblastoma, Neuroblastoma, Oligodendroglioma Pineal astrocytoma, Pituitary adenoma, Visual pathway and hypothalamic glioma.

Breast cancers include Breast cancer, Invasive lobular carcinoma, Tubular carcinoma Invasive cribriform carcinoma, Medullary carcinoma, Male breast cancer, Phyllodes tumour and Inflammatory Breast Cancer.

Endocrine system cancers include Adrenocortical carcinoma, Islet cell carcinoma (endocrine pancreas), Multiple endocrine neoplasia syndrome, Parathyroid cancer Pheochromocytoma, Thyroid cancer and Merkel cell carcinoma.

Eye cancers include Uveal melanoma and Retinoblastoma.

Gastrointestinal cancers include Anal cancer, Appendix cancer, cholangiocarcinoma, Carcinoid tumour, gastrointestinal cancer, Colon cancer, Colorectal cancer, Extrahepatic bile duct cancer, Gallbladder cancer, Gastric (stomach) cancer, Gastrointestinal carcinoid tumour, Gastrointestinal stromal tumour (GIST), Hepatocellular cancer, Pancreatic cancer, islet cell and Rectal cancer.

Genitourinary and gynaecological cancers include Bladder cancer, Cervical cancer, Endometrial cancer, Extragonadal germ cell tumour, Ovarian cancer, Ovarian epithelial cancer (surface epithelial-stromal tumour), Ovarian germ cell tumour, Penile cancer, Renal cell carcinoma, Renal pelvis and ureter, transitional cell cancer, Prostate cancer, Testicular cancer, Gestational trophoblastic tumour, Ureter and renal pelvis, transitional cell cancer, Urethral cancer, Uterine sarcoma, Vaginal cancer, Vulvar cancer and Wilms tumour.

Head and neck cancers include oesophageal cancer, Head and neck cancer, Head and neck squamous cell carcinoma (HNSCC), Nasopharyngeal carcinoma, Oral cancer, Oropharyngeal cancer, Paranasal sinus and nasal cavity cancer, Pharyngeal cancer Salivary gland cancer and Hypopharyngeal cancer.

Skin cancers include Basal-cell carcinoma, Melanoma and Skin cancer (non-melanoma).

Thoracic and respiratory cancers include Bronchial adenomas/carcinoids, Small cell lung cancer, Mesothelioma, Non-small cell lung cancer, Pleuropulmonary blastoma, Laryngeal cancer, Thymoma and thymic carcinoma.

HIV/AIDS related cancers include AIDS-related cancers and Kaposi sarcoma.

In one particularly preferred embodiment, the tumour is primary brain cancer, preferably selected from the group consisting of glioblastoma multiforme (GBM), glioma, diffuse midline glioma, mixed glioma, astrocytoma, oligodendroglioma, medulloblastoma, pineal region tumours, atypical teratoid rhabdoid tumour (AT/RT) and primitive neuroectodermal tumours (PNETS).

In a further particularly preferred embodiment, the tumour is a secondary tumour of any origin, which has metastasized into the brain. In a further particularly preferred embodiment, the tumour is pancreatic ductal adenocarcinoma (PDAC).

The PDAC may be characterised by being a hyaluronic acid-enriched tumour or collagen over-expressing tumour. Such PDACs may be distinguished by biopsy or either RNA or histochemical analysis of the biopsy material, or in some cases through assay of blood serum soluble hyaluronic acid.

In a further particularly preferred embodiment, the tumour is osteosarcoma.

The composition comprises at least one agent which impedes the movement and/or proliferation of cancer cells. In some embodiments, the preventing or inhibiting migration of cancer cells arrests cell or tumour proliferation and induces either differentiation and/or dormancy. Dormancy can include tumour mass dormancy, and/or cellular dormancy.

In tumour mass dormancy, the tumour mass will continue to divide until it is physically limited by size, does not have access to the blood supply, or the immune system acts on it. Here the cells are not completely inactive, but they cannot expand and sit in a balance between proliferation and apoptosis. Tumour mass dormancy is also often associated with angiogenic dormancy. This occurs when tumours enter a hypoxic state because they cannot get to blood vessels. If the number of cells still proliferating is balanced by the number dying from no blood supply, the tumour sits in angiogenic dormancy.

Cellular dormancy refers to the cell entering a state of quiescence where growth is arrested in G0-G1 of the cell cycle, and cells are truly inactive and asymptomatic. This is referred to as the dormancy that tumour cells enter when they survive dissemination but cannot adapt immediately to stresses or the new microenvironment.

Differentiation takes cells, including cancer cells, out of the proliferation cycle and tends to result in a more benign tumour phenotype. Conversely, migratory cancer cell phenotypes are typically associated with de-differentiation and aggressive phenotype [D. (shay-Ronen, M. Diepenbruck, R. K. R. Kalathur, J. Wang C. Hess, G. Christofori, Cancer Cell, 35 (2019) 17].

Cancer cell migration out of or away from the tumour is physically impeded; the space available for cells to multiply is thereby reduced. This results in the prevention or inhibition of the cancer cell migration, and inhibition of the tumour growth. For the tumour to expand, i.e. from cell proliferation and growth, there has to be physical space for the new and growing cells. That space is generated in vivo by cancer cells or cancer-associated cells degrading the surrounding matrix and/or deforming the surrounding matrix, i.e. by compressing it. Crosslinking makes most extracellular matrix molecules more resistant to degradation and deformation. Increasing the molecular density of, e.g. hyaluronic acid or collagens, means that the cells have to break down more material before they can move, proliferate and grow, thus slowing or completely stopping the migration/proliferation processes.

In general, the action of the agent (alone) does not kill the cancer cells or induce apoptosis in the cancer cells.

Preferably, the agent is one which is capable of:
(a) (i) cross-linking cancer cells in the tumour, and/or
   (ii) cross-linking extracellular matrix in and/or around the tumour, and/or
   (iii) cross-linking cancer cells to the extracellular matrix in and/or around the tumour; and/or
(b) increasing the viscosity and/or molecular entanglement of the extracellular matrix in and/or around the tumour or site of the resected tumour, preferably by forming a secondary network in the extracellular matrix in and/or around the tumour.

In one embodiment, the agent is one which is capable of:
(i) cross-linking cancer cells in the tumour, and/or
(ii) cross-linking extracellular matrix in and/or around the tumour, and/or
(iii) cross-linking cancer cells to the extracellular matrix in and/or around the tumour.

Preferably, the agent is a cross-linking agent. Preferably, the agent is one which is capable of performing all of (i)-(iii).

Although the extracellular matrix composition is highly dynamic and specialized to each tissue, in general it consists of collagens and/or other fibrillar/net-forming proteins, hyaluronic acid, and tissue-specific glycoproteins and proteoglycans. Agents which cross-link one or more of these molecules are preferably used.

Preferably, the agent is one which cross-links hyaluronic acid or extracellular matrix proteins or both, including collagens, fibronectin, laminin, extracellular matrix proteoglycans (e.g. aggrecan) and glycoproteins, extracellular proteins expressed by the tumour cells and proteins and other components of exosomes.

In some embodiments, the agent is preferably one which cross-links a proteoglycan which is specific or partially specific to the tissue in which the tumour is located or which is over-expressed in the tumour.

In some embodiments, cross-linking is induced between proteins in the tumour extracellular matrix and surrounding extracellular matrix or between protein and hyaluronic acid. For example, riboflavin plus photoactive light may be used to crosslink collagen and other matrix proteins [E. Spoerl et al, Exp. Eye Res. 66 (1998) 97-103].

In some embodiments, the cross-linking agent is one which is capable of covalently cross-linking chemical groups selected from amines, thiols and carbonyls (including aldehydes, esters, thioesters, carboxylate, ketone and amide functionalities).

Cross-linking agents which are capable of cross-linking amines include carbonyl-containing compounds (e.g. aldehydes and dicarbonyls) and iridoids.

Suitable aldehydes and dicarbonyls include methylglyoxal, glyoxal, glutaraldehyde, oxidized hyaluronic acid, oxidized derivatives of hyaluronic acid and oxidized peptides.

Suitable iridoids include genipin, genipinoside, loganin aglucon, oleuropein aglucon and E-6-O methoxycinnamoylscandoside methyl ester aglucon.

Amines may also be crosslinked to carboxylate or amide groups, forming iso-peptide crosslink bonds. Enzymes such as transglutaminases can catalyse crosslinking between amine and carboxylate/amide groups on existing ECM proteins, or an appropriately designed peptide [B. Zakeri, et al., Proc. Nat. Acad. Sci. USA, 109 (2012) E690] can be used as the crosslinking agent, inducing spontaneous isopeptide bond formation without need of an enzyme.

Cross-linking agents which are capable of cross-linking thiols include polyphenols and quinones. Suitable polyphenols include hydroxylated cinnamic acids such as caffeic acid (3,4-dihydroxycinnamic acid), chlorogenic acid (its quinic acid ester), caftaric acid (its tartaric acid ester), and flavonols such as quercetin and rutin. Also included are catechin, epitcatechin and tannins.

Cross-linking agents which are capable of cross-linking carbonyls in general include polyamines, photo-crosslinkers, e.g. porphyrins, aminolevulinic acid, camphorquinone, fluorescein and riboflavin, eosin Y and compounds containing these molecules as tags. Polyamines include poly-lysine and dihydrazides.

Cross-linking agents which are capable of cross-linking aldehydes include polyamines. Suitable polyamines include dihydrazides, dihydrazine adipic acid, poly-lysine, spermidine and trientine.

Cross-linking agents which are capable of cross-linking esters and thioesters include vinyl ketones in conjunction with a photo-initiator and UV/visible light; compounds that absorb optical wavelength radiation (through single or multiple quantum absorption) in conjunction with an appropriate light source; and ionizing radiation. Suitable vinyl ketones include ethylene glycol dimethacrylate. Suitable photoinitiators include eosin Y and Lucirin-TPO® (BASF).

In some embodiments, extracellular matrix proteins may be crosslinked through hydrogen bonding or hydrophobic interactions or both, using molecules with suitable hydrogen bonding or hydrophobic functional groups. Such molecules include polyamines, polyols, polyphenols, terpenoids (e.g. 8-oxogeranial) and appropriately designed peptides.

Peptides may be used as cross-linking agents to covalently-crosslink protein sidechain amines and carboxylate/amide groups.

Photochemicals with an accessible triplet excited electronic state and which may self-oxidise may be used as cross-linking agents.

The agent may also be an antibody against an extracellular matrix moiety, optionally coupled to a cross-linking agent (e.g. a cross-linking agent as defined above). In some embodiments, the antibody is a bi-specific antibody (e.g. capable of binding to an extracellular matrix moiety and to a tumour-cell specific antigen).

In some embodiments, the cross-linking is enhanced by the use of light, i.e. the cross-linking agent may be a light-induced cross-linking agent. The light may be UV. In some embodiments, the invention includes the step of cross-linking the cross-linking agent using light.

In some embodiments, the cross-linking agent is hyaluronic acid or a derivative thereof.

The derivative may, for example, be oxidized hyaluronic acid (e.g. dialdehyde hyaluronic acid). Hyaluronic acid and such derivatives are capable of diffusing into the ECM around a tumour or the site of a resected tumour; the hyaluronic acid or derivatives thereof may then cross-link ECM proteins (either spontaneously or by initiation with light) or be cross-linked with a separate cross-linking agent.

In some embodiments, the agent induces crosslinking between hyaluronic acid molecules in the extracellular matrix, or between hyaluronic acid and proteins, including glycoproteins and proteoglycans, in the extracellular matrix.

In some embodiments, covalent crosslinking is induced between hyaluronic acid or hyaluronic acid and protein molecules. Agents that induce covalent crosslinking between hyaluronic acid or hyaluronic acid and proteins include glyoxal, methyl glyoxal and glutaraldehyde.

In some embodiments, crosslinking between hyaluronic acid molecules is induced by agents which interact with hyaluronic acid electrostatically (ionically) or through hydrogen bonding. Crosslinking agents that form electrostatic interactions with hyaluronic acid are polycations in general, for example, polyamines such as polylysine, trientine, spermidine, putrescine and polyamine dendrimers such as polyamidoamine (PAMAM) dendrimers. Examples of crosslinking agents that form hydrogen bonds with hyaluronic acid are polyols, such as polyethylene glycol (PEG) and polysaccharides, such as cellulose, starch and chitin.

In some embodiments, crosslinking between hyaluronic acid and proteins may be achieved using molecules that either form covalent bonds or hydrogen bonds with the protein as described above and additionally have functional groups that can form electrostatic or hydrogen bonds with hyaluronic acid. Such crosslinking agents include polyols, including polyphenols and molecules that incorporate functional groups that may covalently bond to proteins as described above and polycation or polyol functionalities that may interact with hyaluronic acid as also described above.

Preferred cross-linking agents include Genipin, Glutaraldehyde, Glyoxal (and derivatives thereof), Proanthocyanidin, Riboflavin (and photoactive light), Fluorescein (and photoactive light), Polyamines, Methylene blue (and photoactive light), trientine, and oxidised hyaluronic acid.

In a further embodiment, the impeding of the movement of the cancer cells is by (b) increasing the viscosity and/or molecular entanglement of the extracellular matrix in and/or around the tumour or the site of a resected tumour, preferably the pre-existing extracellular matrix in and/or around the tumour or the site of a resected tumour. The effect of this is to increase the physical hurdles which the cancer cells must overcome in order to metastasize.

Brain extracellular matrix consists of substantial quantities of hyaluronic acid (a polysaccharide) which forms a highly-hydrated network of molecules, and smaller quantities of specialized proteins, including proteoglycans and linker-proteins (such as the glycoprotein tenascins). The chemical network of hyaluronic acid, linkers and proteoglycan molecules forms a material barrier that cancer cells must displace or degrade in order to proliferate and to migrate.

Increasing the molecular entanglement of this hydrated network prevents cancer cells from displacing the hyaluronic acid and proteoglycan molecules, thus preventing or inhibiting the migration or the growth of the cancer cells.

A similar hyaluronic acid/proteoglycan hydrated network exists in all tissues where these molecular components are present. Increasing molecular entanglement in this network in any tissue may also be effective in preventing and/or inhibiting cancer cell migration.

Preferred embodiments of how this may be done include:
(i) the addition of a polymer which is a component of the extracellular matrix to the site of the tumour; and/or
(ii) the addition of a polymer which is not a component of the extracellular matrix to the site of the tumour.

These polymers may form a secondary network of molecules inside the existing extracellular matrix molecular network in order to reinforce the existing extracellular matrix molecular network. The polymer may itself be cross-linked or not, as desired. The polymer may or may not covalently bind to the existing extracellular matrix molecules without generating covalent crosslinks. The polymer is preferably a non-toxic polymer.

In some embodiments, the polymer is hyaluronic acid or a derivative thereof. Preferably, the hyaluronic acid or a derivative thereof has a molecular weight of 0.5-12 MDa, e.g. 1-3, 3-5, 5-7, 7-9, or 9-12 MDa, more preferably about 1 MDa. In some tumours, hyaluronic acid in the extracellular matrix is degraded or has a lower molecular weight than in healthy tissues, thus reducing the molecular entanglement of the hyaluronic acid present in the cancerous tissue compared to healthy tissue. Addition of higher molecular weight hyaluronic acid, such as hyaluronic acid with molecular weight similar to healthy tissues, may act to increase the molecular entanglement in the cancerous tissue.

Hyaluronic acid may be derivatised to add reactive functional groups by modifying its carboxylic acid groups, its amide group or its hydroxyl (e.g. as reviewed in Khunmanee et al., J. Tissue Engineering, 8 (2017) 1-16).

Examples of modifying hyaluronic acid carboxylic acid groups includes coupling reactions of hyaluronic acid with carbodiimides, such as 1-ethyl-3-(3-dimethyl aminopropyl)-1-carbodiimide hydrochloride (EDC), carbonyldiimidazole or triazoles such as 1-hydroxy-7-azobenzotriazole. These reagents are used to couple hyaluronic acid with compounds containing a crosslinking group as described above (e.g. using carbonyl-containing compounds, including aldehydes and dicarbonyls, polyphenols, amines, iridoids, quinones, vinyl ketones and UV/visible light, compounds that absorb (through single or multiple quantum absorption)).

Modification of the hyaluronic acid amide groups can be through deacetylation, amidation, hemiacetylation, and hemiacetal formation. Deacetylation may be achieved by reaction of hyaluronic acid with anhydrous hydrazine [e.g. Zhang et al., Glycobiology 24, (2014) 1334-42].

Modification of the hyaluronic acid hydroxyl group can be through ether, ester or hemiacetal formation or oxidation. Examples of reagents that are used to react with hyaluronic acid hydroxyl groups (to produce crosslinked hyaluronic acid or hyaluronic acid derivatives with functional groups suitable for subsequent crosslinking to hyaluronic acid or proteins) are divinyl sulfone, octenylsuccinic anhydride, glutaraldehyde, acid chlorides, 1,2,3,4-diepoxybutane, methacrylic anhydride and sodium iodate.

The hyaluronic acid derivative may, for example, be an oxidized hyaluronic acid (dialdehyde hyaluronic acid) containing aldehyde groups formed, for example, by reacting hyaluronic acid with sodium iodate.

Examples of modified hyaluronic acid include amine-modified hyaluronic acid, aminoethylmethacrylated (AEMA)-hyaluronic acid and dialdehyde hyaluronic acid.

The inventors have shown that one such derivative of hyaluronic acid may be used to crosslink extracellular matrix in vivo, and through such crosslinking, provide a physical barrier to cancer cell migration. The inventors have further shown that such derivatives are capable of diffusing into the ECM around a tumour or the site of a resected tumour.

The added hyaluronic acid derivative thereof may then be cross-linked, if desired (to themselves, to other components of the ECM and/or to the cancer cells); this reinforces the structure of the network within the tumour and surrounding extracellular matrix.

Other tissues are collagenous in that the major organic components of their extracellular matrix are collagens, for example the major fibrillar collagens (types I, II and III), the minor fibrillar collagens (types V, XI), the FACIT collagens (types IX, XII, XIV, XIX, XX, XXI and XXIII), the MACIT collagens (types XIII, XXIII and XXV) and the network collagens primarily found in basement membrane (types IV, VIII and X).

Collagenous tissues also contain other proteins such as fibronectin, laminin and proteoglycans as well as some level of hyaluronic add in their extracellular matrix. In some cancers, excess collagen is deposited in and around the tumour, creating local collagen-rich extracellular matrix. In collagenous tissues or in cancers where excess collagen is deposited, cancer cells must degrade or displace extracellular collagens and other proteins in order to migrate and proliferate. Some cancer cells are known to migrate along collagen fibres or fibrils.

In some embodiments, therefore, the polymer is a collagen or a collagen derivative.

The polymer may also be a synthetic polymer such as polylactic add, polyglycolic acid, polyglycolic-co-lactic add. Precursors of these polymers may also be used, with subsequent polymer formation in situ.

These polymers and precursors may also be used to diffuse into the extracellular matrix in and around a tumour site to stiffen the extracellular matrix against cancer cell migration and proliferation.

In yet other embodiments, the viscosity and/or molecular entanglement of the ECM in and/or around the tumour may be increased by contacting all or part of:
(i) the tumour,
(ii) the site of the resected tumour, and/or
(iii) the vicinity of the tumour or site of resected tumour, with a moiety which chemically reacts with one or more components of the ECM, increasing the molecular mass of those ECM components and thereby increasing the viscosity and/or molecular entanglement of the ECM but without crosslinking one or more components of the ECM.

Examples of such moieties include molecules with a single carbonyl group. These will chemically react with terminal amine groups in extracellular matrix proteins and other molecules effectively forming an adduct with those terminal amine groups through a Schiff base-type reaction.

The desired level of viscosity or entanglement of the modified extracellular matrix is preferably that which is equivalent to 4-6 wt % hyaluronic acid of molecular weight 1, 2, 3, 4, 5, 6, 7, 8, 9, 10, 11 or 12 MDa, or greater. The level of entanglement or viscosity is quantifiable relative to a given concentration of hyaluronic acid of known molecular weight using NMR spectroscopy [E. Fischer, P. T. Callaghan, F. Heatley, J. E. Scott, J. Molec. Struct. 602-603 (2002) 303; P. T. Callaghan, Rep. Prog. Phys. 62 (1999) 599].

In some embodiments, the agent additionally comprises a targeting moiety which is specific for the tumour to be removed. For example, the targeting moiety may be an antibody which specifically binds to an epitope on the tumour or tumour matrix in question.

The composition comprising the agent may additionally comprise one or more additional pharmaceutically-acceptable diluents, excipients or carriers.

The composition may comprise one or more agents, as defined herein. For example, the composition may comprise 1, 2, 3 or 4 agents, as defined herein.

For example, the composition may additionally comprise one or more components selected from the group consisting of a buffer, a detergent, an inhibitor of glutathione metabolism (e.g. butionine sulfoximine), a proteinase inhibitor, a metalloprotease inhibitor, a hyaluronase inhibitor, an osmolite (e.g. NaCl, mannitol, etc), and a viscosity modifier.

The composition preferably comprises an effective amount of the agent or agents. As used herein, the term “effective amount” is an amount which is sufficient to prevent or inhibit substantially all (e.g. at least 70%, 80%, 90 or 95%, compared to a control without the agent) of the cancer cells from moving away from the site of the tumour or resected tumour. Effective amounts of each agent may readily be determined by those of skill in the art.

The composition may be applied to all or part of:
(i) the tumour, and/or
(ii) the site of the resected tumour, and/or
(iii) the vicinity of the tumour or site of resected tumour.

The composition may be applied directly or indirectly. For example, the composition may be applied directly in situ. For example, the composition may be applied infusion using a syringe, infusion from a gel or other carrier material, by spraying or by swabbing. Alternatively, the composition may be applied indirectly. For example, the composition may be applied in the vicinity of the tumour (e.g. 1-50 mm from the tumour), wherein the composition diffuses into the tumour or the tumour margins.

Preferably, the composition is applied before, during or after a surgical step to remove (resect) all or part of the tumour. The site of the resected tumour may still contain some cancer cells (the migration of which requires prevention or inhibition).

In some embodiments, the composition is applied before a surgical step to remove all or part of the tumour. In this case, the composition may be administered systemically into the subject, wherein the agent comprises a targeting moiety which is specific for the tumour in question.

In some embodiments, the composition is applied before a surgical step to remove (resect) all or part of the tumour. In this case, the composition may be applied directly to all or part of the tumour and/or all or part of the vicinity of the tumour.

In some embodiments, the composition is applied one or more times during a surgical step to remove (resect) all or part of the tumour. In this case, the composition may be applied directly to all or part of the tumour or all or part of the vicinity of the tumour or the site of the former tumour.

In some embodiments, the composition is applied after a surgical step to remove all or part of the tumour. In this case, the composition may be applied directly to all or part of the former site of the tumour or all or part of the vicinity of the former site of tumour. Alternatively or additionally, the composition may be administered systemically into the subject after removal of the tumour, wherein the agent comprises a targeting moiety which is specific for the tumour to be removed. Preferably, the composition is administered topically into the tumour cavity after removal of the tumour.

During tumour resection, it is not always possible to remove all of the cancer cells. This is particularly a problem in the brain. It is useful therefore to prevent any cancer cells which remain after surgery from continuing to progress or invading further, particularly into the brain.

In one embodiment, therefore, the invention provides an in vivo method of preventing or inhibiting the migration of cancer cells away from the site of a resected tumour on or in a subject, the method comprising contacting all or part of the site of the resected tumour with a composition which impedes the movement of cancer cells.

The composition impedes the movement of the cancer cells, but does not kill them or induce apoptosis.

Preferably, the site of the tumour (or former tumour) is the brain. In one particularly-preferred embodiment of the invention, the tumour is a brain glioblastoma multiforme and the agent cross-links hyaluronic acid, preferably at the former site of the tumour, following resection of all or part of the tumour.

In another particularly-preferred embodiment of the invention, the tumour is a pancreatic ductal adenocarcinoma and the agent cross-links hyaluronic acid or collagen, preferably at the former site of the tumour, following resection of all or part of the tumour. Most preferably, the cross-linking agent is riboflavin (+photoactive light) or genipin.

In another particularly-preferred embodiment of the invention, the agent is oxidized hyaluronic acid (dialdehyde hyaluronic acid), optionally with the addition of a polyamine (for example dihydrazine adipic acid), and the tumour is glioblastoma multiforme.

In another particularly-preferred embodiment of the invention, the agent is riboflavin (+photoactive light), optionally with the addition of a polyamine (for example spermidine or trientine) and the tumour is glioblastoma multiforme.

In another particularly-preferred embodiment of the invention, the agent is a polyamine, (for example, spermidine or trientine) and the tumour is glioblastoma multiforme.

In another particularly-preferred embodiment of the invention, the tumour is an osteosarcoma and the agent cross-links collagen, preferably at the former site of the tumour, following resection of all or part of the tumour. Most preferably, the cross-linking agent is riboflavin (+photoactive light) or genipin.

The disclosure of each reference set forth herein is specifically incorporated herein by reference in its entirety.

### BRIEF DESCRIPTION OF THE FIGURES

FIG. 1: Optical images of U87 tumour spheroids in 3D hyaluronic acid-serum protein model brain ECMs showing the effects of increasing concentrations (i.e. molecular entanglement) of hyaluronic acid on the migration and proliferation of the cancer cells from the initial tumour spheroids. Images on the left of each panel are brightfield images; those on the right show epifluorescence from calcein staining indicating that the cells are metabolically active/alive.

FIG. 2: A: Solution-state ¹H NMR spectra of oxidized riboflavin alone (dashed line), 10% Gibco foetal calf serum alone (solid black line) and a mixture of oxidized riboflavin and 10% Gibco foetal calf serum, in which chemical crosslinking of serum proteins is expected to occur. That chemical reactions have taken place in this mixture is evidenced by the loss of intensity of signals arising from the foetal calf serum, for instance, those indicated by arrows in the figure. B: Optical images of U87 tumour spheroids in 3D hyaluronic acid-serum protein model brain ECMs showing the effect of crosslinking of the serum proteins with riboflavin upon UVA irradiation on cell migration and proliferation against the following controls: (*) 2% hyaluronic acid-serum protein only ECM, (**) 2% hyaluronic acid−serum protein ECM+0.4% riboflavin but no UVA irradiation and (***) 2% hyaluronic acid−serum protein ECM with UVA irradiation. Samples were incubated for 2 days before any UVA irradiation to allow cells to begin to migrate, then the relevant samples irradiated to induce crosslinking. The images are all brightfield images except the bottom row which are epifluorescence spectra of calcein staining (taken in the red channel for samples containing riboflavin to avoid riboflavin fluorescence), indicating that the cells remain metabolically active after matrix crosslinking.

FIG. 3: FT-IR (top) and ¹³C solid-state NMR spectra (bottom) (cross polarization, magic-angle spinning) of hyaluronic acid, oxidized hyaluronic acid (oxHA) (2 hours of sodium iodate reaction) and dihydrazide adipic acid-crosslinked oxidized hyaluronic acid (oxHA-ADH). The black arrow in the oxHA NMR spectrum indicates the signal from the hydrated aldehyde groups induced by the oxidation. The inset in the NMR spectrum of oxHA-ADH shows the signal from Schiff base carbons (—C═N—) as a result of oxHA—adipic acid dihydrazide crosslinking. The spectrum also shows two sharp signals from the adipic acid CH₂ carbons; these signals collectively show that adipic acid has been crosslinked to the oxidized hyaluronic acid.

FIG. 4: Optical images showing the effect of crosslinking oxidized hyaluronic acid (oxHA, hyaluronic acid dialdehyde, see Scheme 1) with adipic acid dihydrazide in 3D hyaluronic acid-serum protein hydrogels on the migration of glioblastoma U87 cell line cells from tumour spheroids compared to controls with no crosslinking (hyaluronic acid—serum model brain ECM, 2 wt % in hyaluronic acid). Images on the left are brightfield images; those on the right are epifluorescence showing calcein staining.

FIG. 5: Optical images showing the effect of diffusing oxidized hyaluronic acid (hyaluronic acid dialdehyde), added as a 2 wt % hydrogel above 3D hyaluronic acid-serum protein model brain ECM (2 wt % in hyaluronic acid), on GBM cancer cell migration. The model brain ECM contains a U87 tumour spheroid in each case. Migration and proliferation of the cancer cells from the spheroids is substantially inhibited compared to controls where non-crosslinkable 2 wt % hyaluronic acid-serum protein hydrogel is added instead of oxHA.

FIG. 6: A. Optical images showing the effect of crosslinking 3D model brain ECM ionically with 5, 10 and 20 mM trientine on the migration of glioblastoma U87 cells from tumour spheroids compared to controls with no trientine crosslinking. Cancer cell migration is inhibited in a trientine-concentration dependent manner. B. Quantification of the extent of cancer cell migration in the 3D model brain ECM with addition of different trientine concentrations. C. Live/dead cell analysis for different trientine concentrations in phosphate-buffered saline shows that trientine is non-toxic to U87 cells, and therefore that inhibition of cell migration with trientine is due to ECM crosslinking and not cell death.

FIG. 7: Optical images showing the effect of crosslinking oxidized hyaluronic acid (oxHA, hyaluronic acid dialdehyde, 2 wt %) with adipic acid dihydrazide on the migration of glioblastoma U251 cell line cells from tumour spheroids compared to controls with no crosslinking (tumour spheroid in hyaluronic acid—serum model brain ECM, 2 wt % in hyaluronic acid). Images on the left are brightfield images; those on the right are epifluorescence showing calcein staining.

FIG. 8: Optical images showing the effect of adding oxidized hyaluronic acid (oxHA, hyaluronic acid dialdehyde) to 3D hyaluronic acid-serum model brain ECM (hyaluronic acid—serum model brain ECM containing 2 wt % in hyaluronic acid) on the migration of glioblastoma U251 cell line cells from tumour spheroids compared to controls (model brain ECM only). OxHA is expected to chemically crosslink serum proteins in the model brain ECM, and that crosslinking to inhibit cancer cell migration. Images on the left are brightfield images; those on the right are epifluorescence showing calcein staining.

FIG. 9: Brightfield optical images of K7M2 mouse osteosarcoma tumour spheroids in 3D collagen-cell culture medium model ECMs showing the effect of crosslinking the ECM proteins with riboflavin upon UVA irradiation on cell migration and proliferation against the following controls: (*) 1.5 mg/ml collagen-medium only ECM models, (**) 1.5 mg/ml collagen-medium only ECM+0.3% riboflavin but no UVA irradiation and (***) 1.5 mg/ml collagen-medium only ECM with UVA irradiation. Samples were incubated for 2 days before any UVA irradiation to allow cells to begin to migrate, then the relevant samples irradiated to induce crosslinking. The images shown are of the samples 11 days after UVA irradiation.

FIG. 10: Brightfield optical images of K7M2 mouse osteosarcoma tumour spheroids in 3D collagen-medium model ECMs showing the effect of crosslinking the extracellular matrix proteins with genipin on cell migration and proliferation against controls (K7M2 tumour spheroids in uncrosslinked 3D 1.5 mg/ml collagen-medium ECMs).

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Development of 3D Model of Brain Tumour

We first developed a 3D in vitro model of glioblastoma multiforme for assessing our crosslinking strategies. The brain extracellular matrix consists of hyaluronic acid, proteoglycans (lecticans (aggrecan, versican, neurocan and brevican) and others (decorin, biglycan, phosphacan)), link proteins, ITIH2 and tenascin-R (along with smaller amounts of other tenascins), fibrous glycoproteins, the most abundant of which are fibronectin and laminin, basement membrane proteins (primarily collagen IV and laminin, also proteoglycans, agrin and perlecan) and notably little fibrous collagen. The composition of the brain extracellular matrix overall by weight is approximately 10% hyaluronic acid: 15% proteoglycans: 1% collagen IV by dry weight [K. Koh, J. Cha, J. Park, J. Choi, S.-G. Kang, P. Kim. Scientific Reports 8 (2018) 4608], corresponding to 3%: 4.5%: 0.3% by fresh weight respectively, assuming 70% of brain tissue is water. The most abundant single component of the brain extracellular matrix is thus hyaluronic acid, and moreover, the next most abundant components, various proteoglycans, contain charged polysaccharide post-translational modifications that physico-chemically mimic hyaluronic acid.

We first established a 3D extracellular matrix (ECM) model for brain consisting of hyaluronic acid mixed with serum proteins to support GBM cell metabolism. We then used tumour spheroids, cultured from U87 GBM cell line cells, as 3D tumour models that we implanted into the hyaluronic acid-serum model brain ECM. Our first aim was to establish whether hyaluronic acid concentration had any effect on GBM cell migration that would suggest that increased molecular entanglement can affect cancer cell migration.

We fabricated hyaluronic acid-serum model brain ECMs containing 2 wt %, 3.5 wt % and 5 wt % hyaluronic acid to represent low, normal and high hyaluronic acid concentrations/levels of molecular entanglement with respect to brain extracellular matrix. The low hyaluronic acid concentration model ECMs can be expected to mimic an in vivo situation where there is substantial hyaluronic acid degradation and the high hyaluronic acid concentration was set to exceed the average hyaluronic acid concentration in brain ECM by 50%. We used 1 MDa molecular weight hyaluronic acid in these model ECMs, to correspond to a typical value within the range of molecular weights found in vivo (˜0.5-2 MDa) [X. Tian, J. Azpurua, C. Hine, A. V. Max Myakishev-Rempe, J. Ablaeva, Z. Mao, E. Nevo, V. Gorbunova, A. Seluanov. Nature 499 (2013) 346-349].

The different concentrations of hyaluronic acid-serum for model ECMs were prepared by mixing hyaluronic acid with MEM complete media and foetal calf serum. A stock 9% hydrogel was prepared by thoroughly mixing 300 mg of hyaluronic acid with 3000 μl of MEM complete media supplemented with 10% foetal calf serum, antibiotics, and pyruvate and left overnight to achieve uniform hydration throughout the hydrogel. Varying weights of hyaluronic acid-serum stock mix were transferred to wells of chamber slides and diluted with MEM complete media to achieve the desired final concentrations of hyaluronic acid as given in the table below, then incubated overnight in 5% CO₂ in a cell culture incubator to allow the gels to settle before use.

| | Weight | | Volume of media |
|---|---|---|---|
| | of 9% | Volume of | transferred together |
| | HA (mg) | Media (μl) | with spheroid (μl) |
| 5 wt % HA gel | 167 | 83 | 50 |
| 3.5 wt % HA gel | 117 | 133 | 50 |
| 2 wt % HA gel | 67 | 183 | 50 |

U87 GBM cells were cultured in MEM complete medium supplemented with 10% foetal calf serum, antibiotics, and pyruvate. The cultures were maintained in T-175 cm² culture flasks and incubated at 37° C. and 5% CO₂ in a humidified incubator. At 70% confluence, cells were detached from the surface of the flasks after incubating for 5 min at room temperature with 10 ml of trypsin-EDTA. The cell suspension was then centrifuged at 1500 rpm for 5 min at room temperature to pellet the cells. The pellet was re-suspended in MEM complete medium, and a cell count was taken using a hemocytometer counting slide, after which the cells were seeded for spheroid formation in 96-well round-bottom ultra-low adhesion microplates at a cell density of 5×10³ cells per well in a final volume of 200 μl of MEM complete medium. The plates were then centrifuged at 1400 rpm for 10 minutes and incubated in a 37° C. and 5% CO₂ in a humidified cell culture incubator. Cell aggregation was noticed immediately after centrifugation and spheroid formation was observed 48-96 hours post-incubation. The spheroids were incubated for a total of 9 days with media changes on day 4 and 7.

Calcein AM (acetoxymethyl), a non-fluorescent ester derivative of the fluorescent calcein dye, was used to determine the viability and integrity of spheroids. Spheroids were soaked in 1 uM calcein-AM in live cell imaging buffer for 45 minutes in a humidified 5% CO₂ incubator. After incubation, the calcein-AM solution was removed, spheroids were washed three times with PBS and transferred with 50 μl of media to the top of hyaluronic acid-serum gels in chamber slide wells prepared as above. In controls, spheroids were placed into the wells with 300 μl of MEM complete supplemented medium.

Migration of cells from the spheroids, cell viability and the vertical position of the spheroid in the well were recorded on days 4, 7 and 16 using bright field, epifluorescence or confocal microscopy, before the samples were returned to the incubator to continue the experiment.

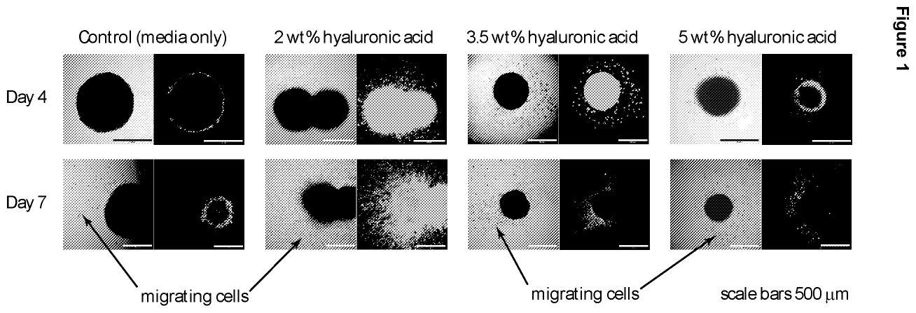

The results are shown in FIG. 1. The U87 cell tumour spheroids sunk into their respective hyaluronic acid-serum model matrices within the first few hours, so were clearly in a 3D environment and in all cases, calcein-AM staining showed that the cells remained metabolically active throughout the period of the experiment. Control spheroids (in media only) (samples, n=8) remained intact over the course of the 16-day experiment. They enlarged in diameter and shed many isolated cells, which spread over the bottom of each well.

Spheroids in the 5 wt % hyaluronic acid—serum model brain ECMs (n=8) remained suspended and never reached the well bottom. Importantly, they showed shrinking of spheroid diameter, probably due to compactization of cell packaging within the spheroid, and no cell migration was observed.

Spheroids in the 2 wt % HA model ECMs (n=8) typically sank to the bottom of the well within 3-7 days, then completely collapsed, releasing lots of cells, which proliferated and migrated rapidly, leading to a thick layer of cells covering the bottom of the well with no clear spheroid structure remaining. Interestingly, this cell response to the low concentration gel was far more extreme than in the control samples of spheroids in media only, where the spheroid remained intact and only isolated cells were shed. Spheroids in the 3.5 wt % model ECMs (n=8) showed two different types of behaviour: five spheroids remained intact, showed some growth and released a few migrating cells whilst three behaved similarly to spheroids in 2 wt % HA model ECMs: they sank to the bottom of the well within 3-5 days, collapsed and showed rapid cell migration and proliferation.

Overall, these experiments allowed us to conclude that ECMs with relatively high hyaluronic acid entanglement, here 5 wt %, inhibits U87 cell proliferation and migration, whilst ECM with relatively low hyaluronic acid entanglement (2 wt %) promotes cell proliferation and migration.

GBM is often accompanied by significant expression of hyaluronidases [C. O. McAtee, J. J. Barycki, M. A. Simpson, Adv. Cancer Res. 103 (2014)1]leading to the expectation that the GBM tumour environment has lower hyaluronic acid concentration than healthy tissue or hyaluronic acid molecules of lower molecular weight. Both result in lower molecular entanglement in the tumour environment, which may facilitate cancer cell migration.

The experimental results show that it is possible to stop or constrain cancer cell migration and proliferation by increasing the degree of molecular entanglement.

### Example 2: Cross-Linking of Serum Proteins

Ways to increase the effective molecular entanglement or viscosity in an ECM include the following:
(i) by crosslinking some of the component molecules, and
(ii) by creating a secondary molecular network within the ECM.

We hypothesised that both of these routes would inhibit cancer cell migration or proliferation. Example 1 showed that the 2 wt % hyaluronic acid—serum model brain ECMs represented a good model in which to trial this hypothesis because this ECM composition strongly favours the cell proliferation and migration that we sought to inhibit. We further noted that serum proteins are expected to be present in the brain tumour environment because of capillary leakage [L. G. Dubois, L. Camanati, C. Righy et al, Cell. Neurosci. 8 (2014) article 418; G. Seano, R. K. Jaln, Angiogenesis, 23 (2020), 9-16]. Thus the presence of serum proteins in the model brain ECM further enhanced the applicability of our model for studying GBM.

We first explored the ECM crosslinking route by crosslinking the serum proteins in 2 wt % hyaluronic acid-serum 3D model brain ECMs using riboflavin and UVA light. Irradiating riboflavin at 365 nm in the presence of oxygen molecules (air) generates singlet oxygen molecules which in turn react with riboflavin generating highly reactive peroxide and dialdehyde products that can readily crosslink protein molecules [Oxidation mechanism of riboflavin destruction and antioxidant mechanism of tocotrienols, Hyun Jung Kim, PhD thesis, Ohio State University (2007)]. Briefly, 0.4% riboflavin, 2 wt % hyaluronic acid—serum model ECMs in the wells of chamber slides were prepared by diluting 67 μg of the 9% stock HA (preparation described in Example 1) with 183 μL of 0.7% riboflavin in MEM complete media and with U87 tumour spheroids generated as in Example 1 added on top in 50 μl of MEM complete media.

The samples were incubated (5% CO₂, 37° C.) for 2 days to allow the spheroids to sink to the bottom of the well and cell migration to begin, then irradiated with 365 nm light for 45 minutes to oxidise riboflavin and induce crosslinking between serum proteins. Migration of cells from the spheroids, cell viability and vertical position of the spheroid in the wells were recorded on days 3, 9 and 11 after UVA irradiation using bright field and epifluorescence microscopy, before the samples were returned to the incubator to continue the experiment, as in Example 1.

Control model ECMs were 2 wt % hyaluronic acid—serum alone, 2 wt % hyaluronic acid—serum with 0.4% riboflavin, but no UVA irradiation (sample kept in the dark) and 2 wt % hyaluronic acid—serum which was irradiated with UVA light but contained no riboflavin; no chemical reactions were expected in any of these.

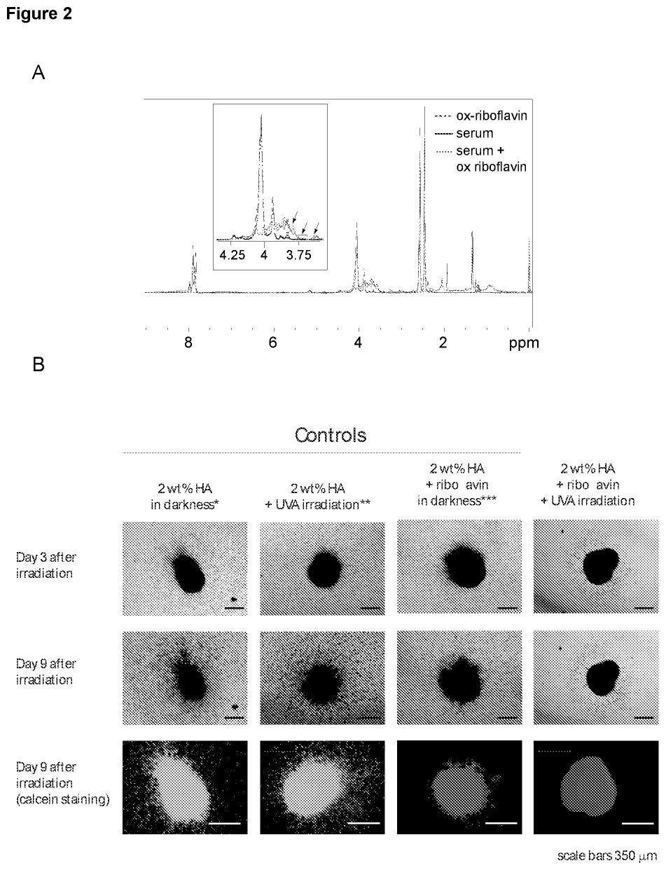

FIG. 2A demonstrates that oxidized riboflavin has reacted with serum proteins. The solution state ¹H NMR spectrum of Gibco foetal calf serum shows a loss of signal intensity in the expected spectral region for peptides/amino acids compared to oxidized riboflavin or serum alone. Examples of NMR signals affected are indicated by arrows in FIG. 2A.

The effect of crosslinking serum proteins in the 2 wt % HA—serum model brain ECM on GBM cell migration are shown in FIG. 2B. The spheroids in the control 2 wt % hyaluronic acid—serum model ECMs behaved as previously (Example 1) with expansion of the spheroids, their gradual disintegration, and large numbers of cells migrating from the spheroids. Similar behaviour was observed in the other controls, showing that riboflavin or UVA irradiation alone did not affect tumour cell migration.

In contrast, in the model ECMs with riboflavin addition and UVA light irradiation, cell migration was effectively halted from the time point at which UVA irradiation initiated crosslinking of the serum proteins in the ECM. Calcein-AM staining showed that the cells remained metabolically active throughout the period of the experiment for all the model ECMs.

### Example 3: Covalent Cross-Linking of the ECM with Oxidized Hyaluronic Acid

As a second demonstration of the effects of crosslinking on cancer cell migration and proliferation, we created model brain extracellular matrices which we crosslinked with oxidized hyaluronic acid. We generated oxidized hyaluronic acid with sodium iodate (product of oxidation, oxHA). In our first experiments, we crosslinked the oxidized hyaluronic acid with adipic acid dihydrazide (ADH) via Schiff base chemistry according to Scheme 1 [W.-Y. Su, Y.-C. Chen, F.-H. Lin, Acta Biomater. 6 (2010) 3044-55] and examined the behaviour of invasive cancer cells in this crosslinked material (plus cell culture medium) alone.

Briefly, hyaluronic acid was dissolved in 100 ml of Milli-Q water to a final concentration of 1 wt % in a beaker and incubated at room temperature overnight, then oxidised by dropwise addition of 15 ml of sodium periodate (2.6%) in PBS while stirring. The reaction mixture was incubated in the dark for 24 hours or 2.5 hours to attain different levels of hyaluronic acid oxidation. The reaction was stopped by the addition of 2.5 ml of 10% ethylene glycol. After oxidation, the hyaluronic acid solution was dialysed using 60,000-80,000 Kd dialysis tubing to remove the oxidation reaction by-products. Milli-Q water was used as the dialysis buffer and resulting oxidised hyaluronic acid (oxHA) was subjected to dialysis for three days with the water replaced twice daily.

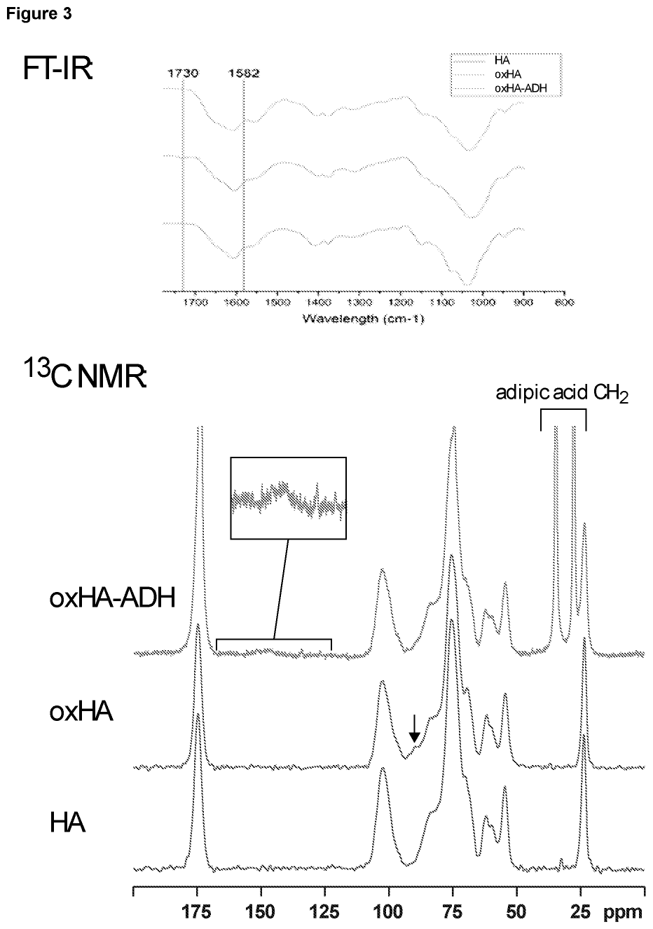

The oxHA solution from the dialysis tubing was then transferred to a 50 ml tube and frozen by immersing the tube in liquid nitrogen, then freeze-dried. To ascertain that addition of adipic acid dihydrazide created the desired crosslinked product, samples of freeze-dried oxHA were crosslinked (product denoted oxHA-ADH) by using 5 wt % adipic acid dihydrazide (ADH) in water. The oxHA-ADH solution was freeze-dried as for oxHA. Solid-state NMR and FTIR spectroscopy were used to verify the composition of the freeze-dried oxHA-ADH crosslinked polymer (FIG. 3).

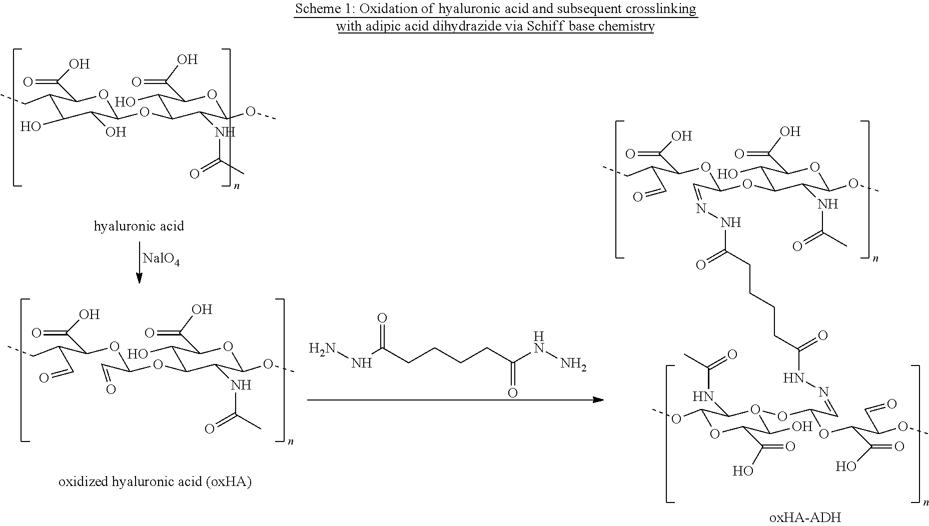

To generate crosslinked oxidized hyaluronic acid, 100 mg of oxHA was dissolved in 800 μl of PBS, mixed thoroughly and incubated overnight. 400 μl of 5 wt % adipic acid dihydrazide was added to the oxHA gel and homogenised using a metal spatula. This was followed by the addition of 1.8 ml of MEM complete media to make a stock crosslinked gel with final concentration of 3 wt % oxHA. 300 mg of the stock gel was transferred to each of the wells of chamber slides and further diluted by adding 100 μl of media to each well, and the resulting oxHA gels allowed to settle for 1 hour at room temperature. Control hyaluronic acid gels were also prepared exactly as for crosslinked gel but by replacing oxHA with hyaluronic acid and adipic acid dihydrazide with an equivalent volume of PBS.

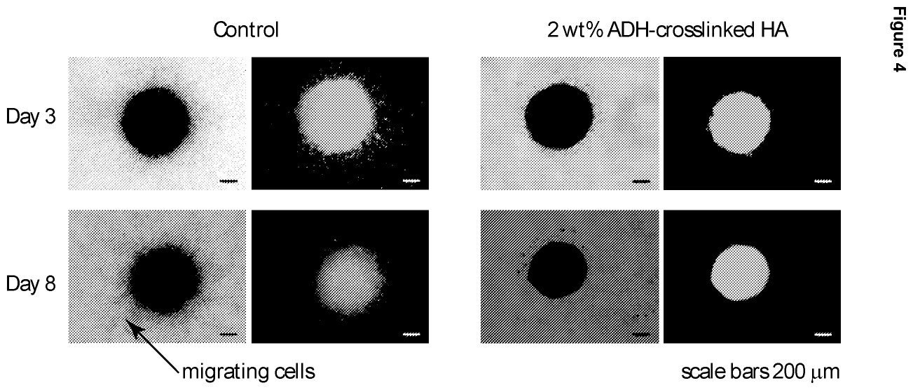

Calcein-stained U-87 tumour spheroids prepared as in Example 1 were transferred into the crosslinked oxHA gels (n=7) and control (n=6) (uncrosslinked, un-oxidized) hyaluronic acid gels along with 50 μl of media to make a final gel concentration of 2 wt % oxHA or hyaluronic acid. The chamber slides were then incubated in a tissue culture incubator (5% CO₂, 37° C.) and imaged at different time points using bright field and epifluorescence microscopy. Results are shown in FIG. 4. Again, calcein staining showed that the cell remained metabolically active in all cases. Tumour spheroids in the control samples behaved as previously with significant cell migration and collapse of the tumour spheroid structure. In contrast, comparatively few cells were observed away from the main tumour spheroids in the crosslinked hyaluronic acid gel samples; those that did migrate from the spheroid appeared to be contained within a small halo around the spheroid.

### Example 4: Diffusion and Crosslinking of Oxidized Hyaluronic Acid (oxHA) in Model Brain ECM

In order to generate crosslinking between extracellular matrix molecules around a tumour in vivo, it will be necessary to introduce suitable crosslinking agents into the tumour region by diffusion. Example 3 shows that oxHA can crosslink molecules with terminal amine groups. The extracellular matrix in all tissues contains an abundance of terminal amine groups in its various proteins. For oxHA to be a suitable crosslinking agent in tumour therapy, it should diffuse from the delivery point in the affected tissue around the tumour and crosslink proteins in that same region. We thus next explored whether oxHA could diffuse into our 3D hyaluronic acid-serum model brain extracellular matrix sufficiently far when added to the top of the 3D model matrix without mixing to crosslink serum proteins around a model tumour sufficiently to inhibit cell migration from that tumour.

Hyaluronic acid-serum model ECMs in MEM complete media were generated and oxHA hydrogels in PBS were generated as described previously to give a final concentration of 2 wt % with respect to the hyaluronic acid component in both cases. 200 mg of the hyaluronic acid-serum model ECM was added to each well of a chamber slide, then a calcein-stained U87 tumour spheroid in 50 μl of MEM complete media added to each well. After the tumour spheroid had settled (typically a few hours), 200 mg of 2 wt % oxHA (prepared as in Example 3) was added to the top of each well. The chamber slide was placed in an incubator (5% CO₂, 37° C.) and the cell migration assessed at day 3 and 8 as previously.

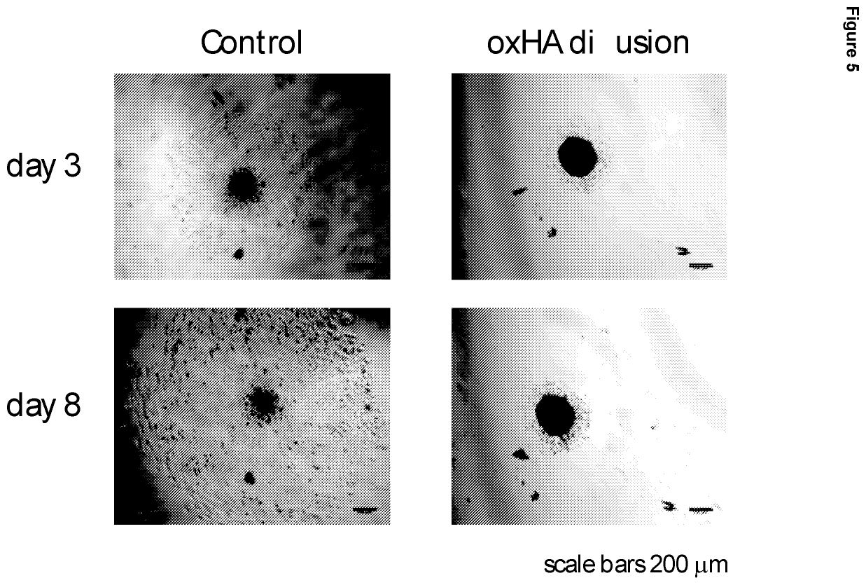

The tumour spheroids (FIG. 5) all sank into the 2 wt % hyaluronic acid-serum model ECM within the first few hours as previously, so that each spheroid was surrounded by ECM that normally promotes cell migration. In the control samples, where 2 wt % hyaluronic acid-serum gel was added above the tumour spheroid instead of oxHA, there was considerable cell migration and proliferation, as expected. Where oxHA was added above the tumour spheroid, however, cell migration was inhibited and consistent with molecular crosslinks having formed around the tumour spheroid. Thus, oxHA must have diffused sufficiently far to generate crosslinking around the tumour spheroid or oxHA encapsulated the tumour spheroid before the spheroid sank into the 2 wt % hyaluronic acid-serum protein model ECM.

### Example 5: Electrostatic Cross-Linking of the ECM

As a third demonstration of how crosslinking in the hyaluronic acid-serum model brain extracellular matrix affects cancer cell migration, we crosslinked the hyaluronic acid component of our model brian ECM electrostatically with a polyamine, trientine, utilising the electrostatic interaction between the trientine positively-charged amine groups and the negatively-charged carboxylate groups of hyaluronic acid to generate crosslinks between hyaluronic acid molecules [D. Ge, K. Higashi, D. Ito, K. Nagano, R. Ishikawa, Y. Terui, K. Higashi, K. Moribe, R. J. Linhardt, T. Toida, Chem. Pharm. Bull. 64 (2016) 390-398]. Trientine is also a drug used to treat Wilson's disease.

Briefly, U87 tumour spheroids were seeded onto either control 2 wt % hyaluronic acid-serum model brain ECMs in chamber slide wells or 2 wt % hyaluronic acid-serum model ECMs with 5, 10 and 20 mM trientine mixed into them, made as described in Example 1, and placed in an incubator (5% CO₂, 37° C.) with cell migration assessed at days 1, 3, 6 and 10.

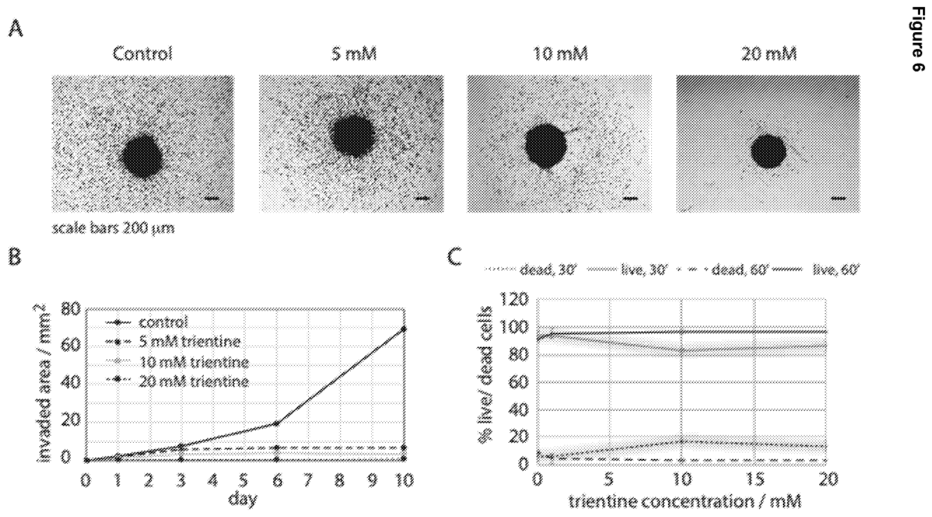

The control samples all showed considerable cell migration as previously whilst cell migration was clearly inhibited in the samples where crosslinking trientine had been added (FIG. 6).

### Example 6: Inhibition of U251 Tumour Spheroids

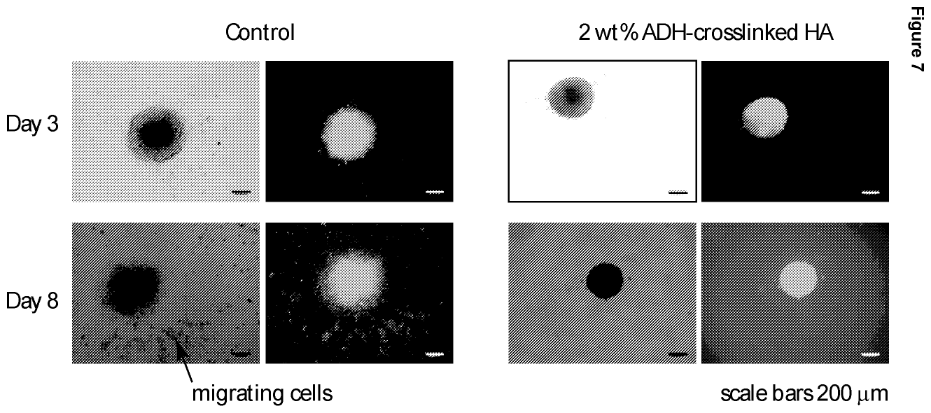

To ascertain the generality of the effects of crosslinking extracellular matrix on brain cancer cell migration, we then performed experiments with a different GBM cell line, U251. In control 2 wt % hyaluronic acid-serum model brain extracellular matrix made up in MEM complete media (FIG. 7, controls), U251 cells show considerable migration of isolated cells, similar to the results for U87 cells on the same concentration hyaluronic acid-serum matrices.

Crosslinking 2 wt % oxidized hyaluronic acid (oxHA) with adipic acid dihydrazide (see above) showed very similar results on U251 cell migration (FIG. 7) as for the U87 tumour spheroids, with very few migrating cells.

In a second demonstration with U251 tumour spheroids, we employed oxHA as an ECM crosslinker in our 2 wt % HA—serum model brain ECM. Briefly, hyaluronic acid—serum model brain ECMs were synthesised as in Example 1 using MEM complete media at a concentration to give a final concentration of 2 wt % hyaluronic acid. OxHA was made up as a gel in phosphate-buffered saline (PBS), again at a concentration to give a final concentration of 2 wt % oxHA and then mixed in 1:1 ratio by weight with the hyaluronic acid—serum model ECM. 400 mg of the resulting mixture was dispensed into each of the wells of a chamber slide and a U251 tumour spheroid stained with calcein in 50 μl of MEM complete media as in Example 1 was added to each well. The chamber slides were then placed in an incubator (5% CO₂, 37° C.) and cell migration was assessed at day 3 and 8 as previously. Controls contained 400 mg of 2 wt % hyaluronic acid-serum model brain ECM only.

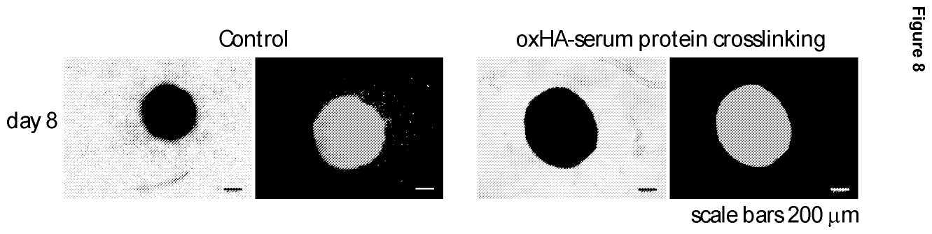

The results are shown in FIG. 8. Calcein staining showed that the U251 cells remained metabolically active throughout the experiment. Once again, the control samples showed considerable cell migration and proliferation, whilst in the samples containing crosslinked hyaluronic acid-serum proteins showed essentially no cell migration even after 8 days of incubation.

### Example 7: Extracellular Matrix Crosslinking to Treat Bone Cancer

To ascertain the universality of the extracellular matrix crosslinking approach to treat solid tumours in general, we next explored the approach in osteosarcoma (bone cancer).

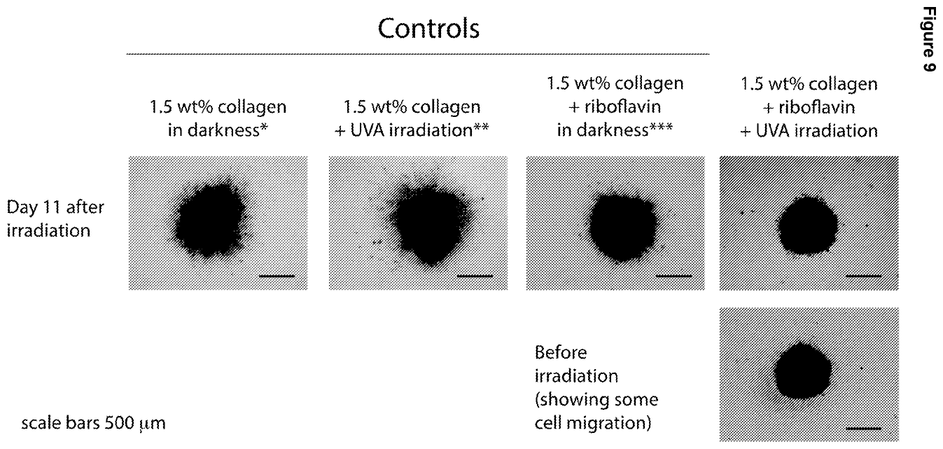

Bone extracellular matrix consists primarily of calcified collagen type I. Calcification is invariably disrupted around metastasizing osteosarcoma tumours, allowing tumour cells to migrate through the extracellular matrix. Thus, we chose to model the bone/osteosarcoma extracellular matrix with a 3D 1.5 mg/ml collagen gel. This concentration of collagen gel promoted invasion by the K7M2 mouse osteosarcoma cell line (FIG. 9). We explored the effect of crosslinking the extracellular matrix in this context by the addition of either exogenous genipin alone or riboflavin and subsequent irradiation with UV light as in Example 2.

Collagen gels (1.5 mg/ml) in DMEM complete medium as model bone extracellular matrix was prepared from a stock 5 wt % collagen gel (IBIDI, Germany). K7M2 cells were cultured in DMEM complete medium supplemented with 10% foetal calf serum, antibiotics, and pyruvate. The cultures were maintained in T-75 cm² culture flasks and incubated at 37° C. and 5% CO₂ in a humidified incubator. At 70% confluence, cells were detached from the surface of the flasks after incubating for 2 min at room temperature with 4 ml of trypsin-EDTA. The cell suspension was then centrifuged at 1400 rpm for 5 min at room temperature to pellet the cells. The pellet was re-suspended in DMEM complete medium, and a cell count was taken using a hemocytometer counting slide, after which the cells were seeded for spheroid formation in 96-well round-bottom ultra-low adhesion microplates at a cell density of 5×10³ cells per well in a final volume of 100 μl of DMEM complete medium. The plates were then centrifuged at 4300 rpm for 5 minutes and incubated in a 37° C. and 5% CO₂ in a humidified cell culture incubator. Cell aggregation was noticed immediately after centrifugation and spheroid formation was observed—24 hours post-incubation. The spheroids were incubated for a total of 7 days.

Briefly, 0.3% riboflavin, 1.5 mg/ml collagen—media gels in the wells of chamber slides were prepared by mixing 100 uL 10xDMEM, 25-30 uL 0.5M NaOH, 20 uL 7.5% NaHCO₃, 655 uL DMEM supplemented with 10% foetal bovine serum and antibiotics and 450 uL of collagen 5 mg/ml stock solution. Riboflavin was pre-mixed into collagen stock solution to achieve final concentration 0.3% in gel. Mixing was performed on ice to avoid premature gelation, pH was checked and adjusted to 7-8 with 0.5M NaOH if found to be too acidic. 250 uL of the resulting mixture was added into each well of a IBIDI slide, moved to room temperature, and 1 spheroid in 50 uL of DMEM supplemented medium per well added immediately. Slides were then moved to a 37° C. (5% CO₂) incubator to initiate gelation of the model ECM. After 30 min gelation manifests itself: the mixture in wells becomes cloudy and thin threads of collagen fibres become visible under the microscope.

The samples were incubated (5% CO₂, 37° C.) for 2 days to allow cell migration from the spheroids to begin, then irradiated with 365 nm light for 45 minutes to oxidise riboflavin and induce protein crosslinking (collagen and serum proteins). Migration of cells from the spheroids, were recorded on days 3, 9 and 11 after UVA irradiation using bright field microscopy, before the samples were returned to the incubator to continue the experiment, as in Example 2.

Control gels were 1.5 mg/ml collagen—medium model ECM alone, 1.5 mg/ml collagen—medium ECM with 0.3% riboflavin, but no UVA irradiation (sample kept in the dark) and 1.5 mg/ml collagen—medium ECM which was irradiated with UVA light but contained no riboflavin; no chemical reactions were expected in any of these.

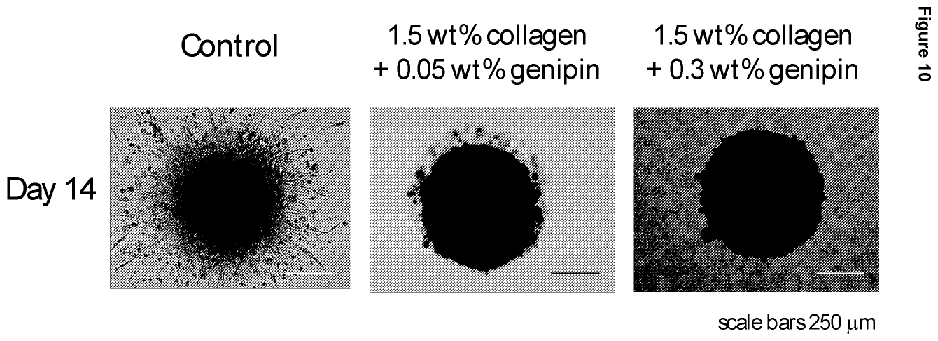

The results are shown in FIG. 10.

We then tested the effect of a different protein crosslinker, genipin (Scheme 2) in the same 3D collagen gel model extracellular matrix.

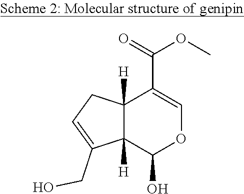 [SMILES: COC(=O)C1=CO[C@@H](O)[C@@H]2C(CO)=CC[C@H]12]

Genipin is a chemical compound found in Genipa americana and Gardenia jasminoides fruits. It is an aglycone derived from an iridoid glycoside called Geniposide. Genipin is an excellent natural cross-linker for proteins and its ability to crosslink collagen has been previously demonstrated [Ko, C. S., Wu, C. H., Huang, H. H., & Chu, I. M. (2007) Journal of Medical and Biological Engineering, 27(1), 7-14; Siriwardane, M. L., Derosa, K., Collins, G., & Pfister, B. J. (2014) Biofabrication, 6(1); Pinheiro, A., Cooley, A., Liao, J., Prabhu, R., & Elder, S. (2016) Journal of Orthopaedic Research, 34(6), 1037-1046; Fessel, G., Gerber, C., & Snedeker, J. G. (2012) Journal of Shoulder and Elbow Surgery, 21(2), 209-217]. It has a low acute toxicity [Fessel, G., Cadby, J., Wunderli, S., Van Weeren, R., & Snedeker, J. G. (2014) Acta Biomaterialia, 10(5), 1897-1906], with LD50 i.v. 153 mg/kg in mice [https://pubchem.ncbi.nlm.nih.gov]. Genipin forms irreversible crosslinks with proteins and glucosamine-containing polysaccharides via nucleophilic attack by protein/glucosamine primary amines groups on the genipin and nucleophilic substitution at the genipin ester group forming a secondary amide [Butler, M. F., Ng, Y.-F., Pudney, P. D. A. (2013) Journal of Polymer Science A, 41, 3941-3953].

Collagen-media gels modelling bone extracellular matrix and K7M2 spheroids were produced as above in the riboflavin/UV light crosslinking study. K7M2 spheroids were grown for 7 days in liquid medium, then seeded into 1.5 mg/ml collagen gel pre-mixed with 1 wt %, 0.3 wt % and 0.05 wt % genipin. Stock genipin solution was 10 wt % in DMSO and diluted with water. Genipin-containing collagen model ECMs were prepared as follows: 100 uL 10xDMEM, 25-30 uL 0.5M NaOH, 20 uL 7.5% NaHCO₃, 150 uL of genipin stock solution & mixed with water or 100% DMSO &r mixed with water to achieve a gel with final concentrations: 1%, 0.3% or 0.05% genipin and 6%, 2% or 0.33% DMSO as corresponding controls, 505 uL DMEM supplemented with 10% foetal bovine serum and antibiotics and 450 uL of collagen 5 mg/ml stock solution. Mixing was performed on ice to avoid premature gelation, pH was checked and adjusted to 7-8 with 0.5M NaOH if found too acidic. 250 uL of mixture was added into each well of an IBIDI slide, moved to room temperature and 1 spheroid in 50 uL of DMEM supplemented medium per well was added immediately. Slides were then moved to 37° C. incubator to initiate gelation of the model ECM. An additional control was K7M2 spheroids seeded onto 1.5 mg/ml collagen—medium model ECMs alone as in the riboflavin/UV irradiation study above.

The collagen-genipin crosslinking reactions result in intense dark blue colouration of the collagen-medium gels due to the presence of air and oxygen radical-induced polymerization of genipin [Butler, M. F., Ng, Y.-F., Pudney, P. D. A. (2013) Journal of Polymer Science A, 41, 3941-3953].

Cell invasion was monitored for 14 days (FIG. 10). There was no observable cell invasion of the model collagen extracellular matrix when crosslinked with 0.3 wt % genipin and only minimal invasion with 0.05 wt % genipin. In corresponding controls (2 wt % and 0.33 wt % DMSO) invasion was not affected by DMSO. The colour intensity in model ECMs with 1 wt % genipin was so intense that observation of spheroid under light microscope was impossible.

The results taken together show that crosslinking extracellular matrix collagen can inhibit the invasion of bone cancer cells into that extracellular matrix to a high degree.

All the Examples above, when taken together, show that cross-linking strategy to impede cancer cell movement, stop invasion and thus metastasis is universal and applicable to a broad range of extracellular matrices regardless of being enriched in glycosaminoglycans or proteins.

### Example 8: Implementation of Cross-Linking Strategies During Surgery

During surgery, the tumour is removed according to best practise for the tumour type and stage. After resection, the chosen crosslinking agent is applied topically to the tumour bed/resection site.

For photo-induced riboflavin crosslinking, riboflavin solution is sprayed into the tumour bed and left to diffuse for a predetermined period of time (minutes per millimetre of depth of penetration required), after which UV irradiation for a prescribed length of time is applied either to the entire tumour bed or to specific sites as determined by the surgeon. Fluorescence from the resulting cross-linked tumour bed ECM or MRI is used to assess the degree and depth of crosslinking achieved.

For crosslinking with oxidized hyaluronic acid, polyols, polyamines and other polycations, the appropriate crosslinking agent is mixed with a (preferably non-toxic) carrier from which it can diffuse, such as hyaluronic acid with molecular weight similar to that of healthy tissue, in a pre-determined ratio, along with broad spectrum antibiotics and a solution/gel formed by addition of PBS to the mixture. The tumour resection cavity is then filled with the resulting solution/gel using a wide gauge Hamilton syringe or similar. Alternatively, a gel is formed at a higher wt % of the carrier, e.g. hyaluronic acid, and crosslinking agent than the final desired formulation and is formed into microspheres. The microspheres are then mixed with a pre-determined quantity of PBS or haemostatic saline and the resulting slurry is immediately poured into the tumour resection cavity.

1. (canceled)

2. A method of preventing or inhibiting the migration of cancer cells away from the site of a tumour or site of a resected tumour on or in a subject, the method comprising contacting all or part of:
(i) the tumour,
(ii) the site of the resected tumour, and/or
(iii) the vicinity of the tumour or site of resected tumour,
with a composition comprising an agent which impedes the movement and/or proliferation of cancer cells, thus preventing or inhibiting migration of cancer cells away from the site of the tumour or site of the resected tumour.

3. (canceled)

4. The method of claim 2, wherein the tumour is selected from the group consisting of a carcinoma, sarcoma, germ cell tumour or a blastoma.

5. The method of claim 2, wherein the tumour is selected from the group consisting of a brain or nervous system cancer, breast cancer, cancer of the endocrine system, eye cancer, gastrointestinal cancer, genitourinary or gynaecological cancer, head and neck cancer, skin cancer, thoracic or respiratory cancer, and a HIV/AIDS-related cancer.

6. The method of claim 2, wherein the tumour is a primary brain cancer.

7. The method of claim 2, wherein the tumour is a pancreatic ductal adenocarcinoma (PDAC) or an osteosarcoma.

8. The method of claim 2, wherein the agent is one which is capable of:
(a) (i) cross-linking cancer cells in the tumour, and/or
(ii) cross-linking extracellular matrix in and/or around the tumour, and/or
(iii) cross-linking cancer cells to the extracellular matrix in and/or around the tumour; and/or
(b) increasing the viscosity and/or molecular entanglement of the extracellular matrix in and/or around the tumour or site of the resected tumor.

9. The method of claim 2, wherein the agent is a cross-linking agent.

10. The method of claim 9, wherein the cross-linking agent is one which is capable of cross-linking a component of the extracellular matrix (ECM) around the tumour.

11. The method of claim 9, wherein the cross-linking agent is one which is capable of cross-linking a chemical group selected from amines, thiols and carbonyls including aldehydes, esters, thioesters, carboxylate, ketone and amide functionalities.

12. The method of claim 9, wherein the cross-linking agent is selected from the group consisting of Genipin, Glutaraldehyde, Glyoxal and derivatives thereof, Proanthocyanidin, and Riboflavin, Fluorescein, Polyamines, Methylene blue, trientine, and oxidised hyaluronic acid.

13. The method of claim 9, wherein the cross-linking agent is a photoactivatable agent which is activatable or subsequently activated by light.

14. The method of claim 2, wherein the agent comprises an antibody.

15. The method of claim 8, wherein the agent is:
(i) a polymer which is a component of the extracellular matrix at the site of the tumour; or
(ii) a polymer which is not a component of the extracellular matrix at the site of the tumour,
and wherein a secondary network is formed of the polymer in the extracellular matrix in and/or around the tumour; or
(iii) a moiety which chemically reacts with one or more components of the ECM to increase the viscosity and/or molecular entanglement of the ECM but without crosslinking one or more components of the ECM.

16. The method of claim 15, wherein the polymer is selected from the group consisting of hyaluronic acid or a derivative thereof, collagen or a derivative thereof, polylactic acid, polyglycolic acid, and polyglycolic-co-lactic acid or precursors thereof.

17. The method of claim 2, wherein:
(i) the composition is applied before a surgical step to remove all or part of the tumour, wherein the agent comprises a targeting moiety which is specific for the tumour;
(ii) the composition is topically applied before a surgical step to remove (resect) all or part of the tumour;
(iii) the composition is applied one or more times during a surgical step to remove (resect) all or part of the tumour;
(iv) the composition is applied after a surgical step to remove all or part of the tumour; or
(v) the composition is applied after a surgical step to remove all or part of the tumour, wherein the agent comprises a targeting moiety which is specific for the tumour to be removed.

18. An in vivo method of inducing dormancy or differentiation in cancer cells in a tumour or site of a resected tumour on or in a subject, the method comprising contacting all or part of:
(i) the tumour,
(ii) the site of the resected tumour, and/or
(iii) the vicinity of the tumour or site of resected tumour,
with a composition comprising an agent which impedes the movement and/or proliferation of cancer cells, thereby inducing cancer cell dormancy or tumour dormancy, or cancer cell differentiation in the cancer cells or the tumour, wherein the agent is as claimed in claim 9.

19. The method of claim 6, wherein the tumour is a primary brain cancer selected from the group consisting of glioblastoma multiforme (GBM), glioma, diffuse midline glioma, mixed glioma, astrocytoma, oligodendroglioma, medulloblastoma, pineal region tumours, atypical teratoid rhabdoid tumour (AT/RT) or a primitive neuroectodermal tumour (PNET).

20. The method of claim 8, wherein the agent is one which is capable of:
(b) increasing the viscosity and/or molecular entanglement of the extracellular matrix in and/or around the tumour or site of the resected tumour, by forming a secondary network in the extracellular matrix in and/or around the tumour.

21. The method of claim 10, wherein the component of the ECM is selected from the group consisting of hyaluronic acid, collagen, fibronectin, laminin, an ECM proteoglycan, an ECM glycoprotein, an extracellular protein expressed by the cancer cells, and a protein or other component of an exosome.

22. The method of claim 17, wherein
(i) the composition is applied before a surgical step to remove all or part of the tumour, wherein the composition is administered systemically into the subject, wherein the agent comprises a targeting moiety which is specific for the tumour;
(ii) the composition is topically applied before a surgical step to remove (resect) all or part of the tumour;
(iii) the composition is applied one or more times during a surgical step to remove (resect) all or part of the tumour;
(iv) the composition is applied after a surgical step to remove all or part of the tumour, wherein the composition is administered topically into the tumour cavity after removal of the tumour; or
(v) the composition is applied after a surgical step to remove all or part of the tumour, wherein the composition is administered systemically into the subject after removal of the tumour, wherein the agent comprises a targeting moiety which is specific for the tumour to be removed.
